(19) United States
(12) Patent Application Publication
(10) Pub. No.: US 2018/0020699 A1
(43) Pub. Date: Jan. 25, 2018

(54) CBD-CONTAINING BEVERAGE

(71) Applicant: THC PHARM GMBH THE HEALTH CONCEPT, Frankfurt Am Main (DE)

(72) Inventor: Christian STEUP, Hofheim (DE)

(21) Appl. No.: 15/536,884

(22) PCT Filed: Dec. 21, 2015
(86) PCT No.: PCT/EP2015/080894

§ 371 Date: Jun. 16, 2017

(30) Foreign Application Priority Data
Dec. 19, 2014 (EP) 14199135.6

(51) Int. Cl. A23L 2/52; A61K 36/185; A61K 31/05; A61K 31/352; A23L 33/105; A23L 35/00

(52) U.S. Cl. CPC A23L 2/52; A23L 33/105; A23L 35/10; A61K 31/05; A61K 31/352; A61K 36/185

(57) ABSTRACT

The invention relates to a CBD-containing liquid formulation, and in particular to a beverage comprising at least one emulsifier.

The invention relates to a CBD-containing liquid formulation, and in particular to a beverage comprising at least one emulsifier.

Cannabidiol (CBD for short) ((2-[1R-3-methyl-6-(1 methylethenyl]-2-cyclohexen-1-yl]-5-pentyl-1,3-benzenediol)) is a mildly psychoactive cannabinoid made from female hemp Cannabis sativa/indica. CBD has an anticonvulsant, anti-inflammatory, anxiolytic effect and is effective against nausea (overview in: Zuardi AW, Cannabidiol: from an inactive cannabinoid to a drug with wide spectrum of action, Rev Bras Psiquiatr, 2008 Sep;30(3):271-80).

CBD chemical formula

A variety of administration forms and formulations for CBD are described in the prior art, and the application thereof, such as in EP1289517B1, WO2008019146; however, all of these relate to an at least partially solid formulation. Furthermore, it is known that CBD in aqueous solution causes a very bitter taste.

It is the object of the present invention to provide a liquid formulation, which can be administered as a beverage, for example, to a human or an animal.

The object is achieved by claim 1.

A further object relates to the provision of CBD-containing liquid formulations comprising a considerably elevated amount of CBD, which are nonetheless palatable, and do not have the bitter CBD taste, and can be orally administered.

The invention thus relates to CBD-containing beverages, base materials, methods for productions, and the use thereof.

Surprisingly, a beverage in which CBD is dissolved in a stable manner in very high concentrations, and which also does not precipitate when stored or kept available for extended periods, is obtained when CBD is combined with at least one emulsifier.

The emulsifier is particularly preferably selected from the group consisting of polysorbates, such as commercially available polyoxyethylene sorbitan fatty acid ester (Tween) and Span products, in particular Tween 20, 60, 80, rubbers such as gum arabic, pectins, starches and modified starches, such as Purity Gum, and/or proteins, such as caseinates from milk.

In a preferred embodiment, the emulsifier is a purified emulsifier or a mixture of emulsifiers, which essentially does not comprise further substances. According to this preferred embodiment, the emulsifier is not a component of a mixture of substances which comprises emulsifiers, such as milk powder or powdered milk.

Emulsifiers can advantageously furthermore be selected from the group of non-ionic, anionic, cationic or amphoteric emulsifiers.

The non-ionic emulsifiers used can be different emulsifiers from the group consisting of partial fatty acid esters, fatty alcohols, sterols, polyethylene glycols, such as ethoxylated fatty acids, ethoxylated fatty alcohols, and ethoxylated sorbitan esters, sugar emulsifiers, polyglycerol emulsifiers, and silicon emulsifiers.

The anionic emulsifiers used can be different emulsifiers from the group consisting of soaps, such as sodium stearate, fatty alcohol sulfates, mono, di- and tri-alkyl phosphoric acid esters and the ethoxylates thereof, fatty acid lactate esters, fatty acid citrate esters, and fatty acid citroglycerin esters.

Cationic emulsifiers can be, for example, quaternary ammonium compounds having a long-chain aliphatic group, such as distearyldimonium chloride.

Amphoteric emulsifiers can include different emulsifiers from the groups consisting of alkylamininoalkane carboxylic acids, betaines, sulfobetaines, or imidazoline derivatives.

According to the invention, furthermore naturally occurring emulsifiers are preferred, which include, for example, beeswax, lecithin and sterols, which can likewise be used for producing a liquid formulation according to the invention.

The invention thus relates to a liquid formulation comprising cannabidiol and water and at least one emulsifier.

According to the invention, the CBD-containing beverage is preferably produced by introducing the CBD in the form of a stable emulsion into the finished beverage or the corresponding beverage base material. The finished beverage does not have to be an emulsion, even though those are likewise covered by embodiments according to the invention. Emulsifiers and water can be added in arbitrary amounts to the cannabidiol.

In a preferred embodiment, the CBD is preferably produced synthetically, in keeping with the teaching of the applicant from EP 2314580 A1. The CBD can likewise also be obtained from cannabis extracts and used after purification (see, e.g., DE10051427C1).

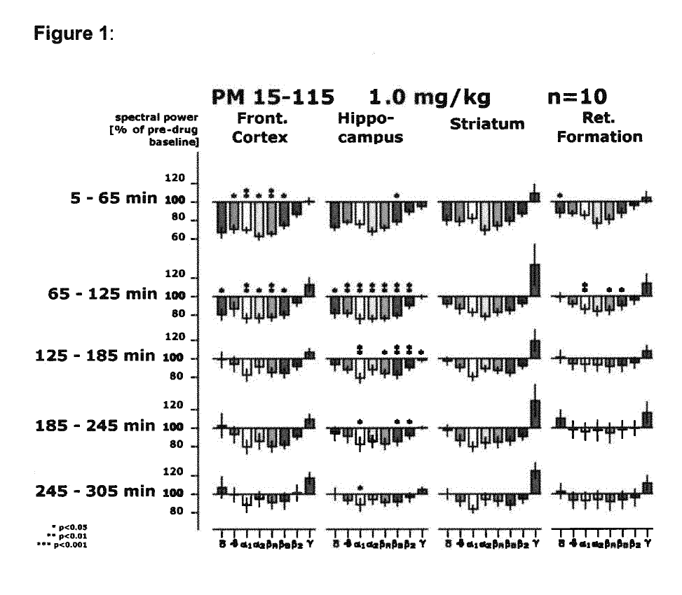

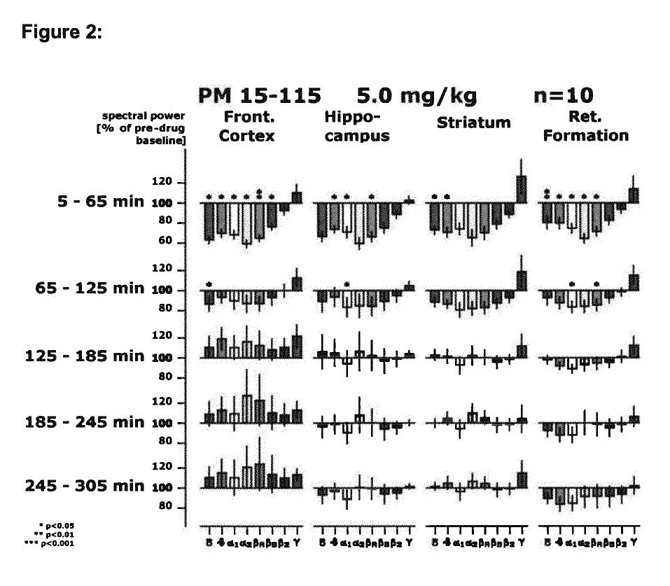

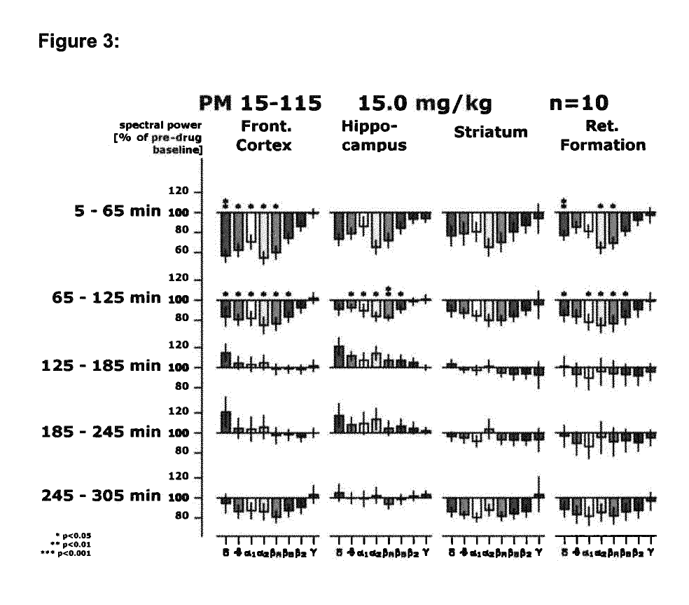

FIGS. 1 to 3 show electropharmacograms from electroencephalography (EEG) of adult rats (>3 months old), wherein a dosage of 1 mg/kg (body weight) (FIG. 1), 5 mg/kg (body weight (FIGS. 2), and 15 mg/kg (body weight) (FIG. 3) cannabidiol was administered in the form of a liquid formulation.

Under general anesthesia, four depth electrodes were implanted into the prefrontal cortex, the hippocampus, the striatum, and the reticular formation of the rats. From the head of the rats, signals are wirelessly transmitted by radio to a computer. At the same time, the physical activity is measured.

The signals are subjected to a frequency analysis, for example using the fast Fourier transform (FFT), and the frequency content of a signal is determined. In the case of EEG data, the spectrum is divided into multiple regions, which historically are denoted by delta, theta, alpha and beta.

After the division into seven frequency ranges ((1) to (7), see FIGS. 1-3), the changes in the electric output after administration of a liquid formulation according to the invention comprising cannabidiol can be quantified and, taking the signals of the four brain regions into consideration, a specific change pattern, referred to as the “electropharmacogram (supra)”, is obtained (Dimpfel W., Pharmacological classification of herbal extracts by means of comparison to spectral EEG signatures induced by synthetic drugs in the freely moving rat. Journal of Ethnopharmacology 2013, 149, 583-589).

An increase in the gamma spectral power (7) (see FIG. 1) can be established at a dosage of 1 mg/kg to 5 mg/kg within the striatum, to a lesser degree in the reticular formation and prefrontal cortex, and not at all in the hippocampus. This specific increase can be observed for at least 4 hours. Gamma spectral power correlates with increased activity (Dimpfel W (2015) Drug Discovery and Translational Medicine. Neurophysiological Techniques Provide a Holistic Approach to Saving Animals. BoD Verlag Norderstetten). In contrast, no specific increase in gamma spectral power is established for a dosage of 15 mg/kg GED (FIG. 3).

As a result, high dosages of cannabidiol in a liquid formulation, and more particularly 5 or 15 mg/kg (body weight) according to FIGS. 2 and 3, have a sedating effect. The EEG pattern or electropharmacogram corresponds to that of certain antidepressants and analgesic drugs. This is confirmed by the significant decrease in the activity index. At the lowest dosage, and in particular less than 5 mg/kg (body weight), less than 2 mg/kg (body weight), and preferably 1 mg/kg (body weight), an activating and invigorating effect was observed.

A preferred embodiment of the invention thus relates to a liquid formulation comprising cannabidiol and water and at least one emulsifier, wherein a dosage of 1 mg/kg to 5 mg/kg (body weight) CBD is administered/applied to a human or an animal.

In a preferred embodiment, a liquid formation can thus comprise up to 500 mg/L CBD, 300 mg/L CBD, 100 mg/L CBD, preferably 40 mg/L CBD, 50 mg/L CBD, 60 mg/L CBD and in particular 20 mg/L CBD, 10 mg/L CBD. Furthermore, a range of 50 mg/L to 300 mg/L CBD, and in particular 50 mg/L to 100 mg/L CBD, in a formulation according to the invention is preferred. The administration of 60 mg/L CBD of a liquid formulation, for example, allows a dosage of 1 mg/kg (body weight) at a body weight of 60 kg.

Positive properties that a corresponding beverage yields include a performance-enhancing, invigorating, agreeable, pleasant, but not psychoactive effect for humans or animals. These effects can already develop starting at 10 mg/L CBD in a liquid formulation.

Further positive effects include increased concentration, increased cognitive ability, and increased memory function.

The invention thus relates to a product, and in particular a pharmaceutical product or a dietary supplement in a liquid formulation comprising cannabidiol and water and at least one emulsifier for use and application to increase concentration, to increase physical, athletic and cognitive performance, and to increase memory function, if necessary along with auxiliary substances and additives.

The invention thus relates to an energy drink, comprising a liquid formulation comprising cannabidiol and water and at least one emulsifier, along with auxiliary substances and additives.

Due to the unexpected positive properties, the present invention considers, as a further embodiment, the option that the formulation or the beverage is present as mineral water, lemonade, tonic water, a sports, mineral, fruit, fruit juice, milk, whey or soft drink or an alcoholic beverage, such as beer, or as a drinking water preparation. In the broadest sense, the present invention relates to a life style product, and in particular to a product for enhancing performance.

Moreover, the invention likewise relates to a flavoring agent comprising cannabidiol in a liquid formulation. Such a flavoring agent according to the invention can be used as a quinine substitute, for example. The invention thus relates to a flavoring agent, comprising a liquid formulation comprising cannabidiol and water and at least one emulsifier.

In a further embodiment, the liquid formulation or the beverage can be mixed with flavor components. These flavor components are preferably used to mask the bitter taste, and a person skilled in the art will be able to provide corresponding flavor components. Examples of flavor components are preferably, without being limited to, food flavoring agents, as described in Regulation (ED) No. 1334/2008. In particular, fruit flavorings are preferred, such as lemon, strawberry and the like.

The present invention likewise considers the use of the claimed liquid formulation as a physiological restorative agent, and in this connection in particular in the form of a foodstuff or food supplement (see, e.g., without being limited thereto, EC Directive 2002/46/EC of Jun. 10, 2002), optionally comprising auxiliary substances and additives, or a functional food for humans or animals.

Additional advantages and characteristics of the present invention will be apparent from the description of exemplary embodiments. The following examples and figures are provided to describe the invention, without limiting the invention to these examples.

### EXAMPLES

20% (w/w) CBD is dissolved in Polysorbate 80 (heat in ultrasonic bath for approximately 1 hour, if necessary in a water bath). Thereafter, 0.1 g of the clear CBD solution is added to 0.5 L Sprite® (=lemon-flavored soft drink) using a pipette. Should the CBD solution not be clear, this must continue to be dissolved in the ultrasonic bath or optionally heated in a water bath. Initially, minor turbidity of the drinking solution on the surface develops. The bottle is closed and placed for approximately 45 seconds into an ultrasonic bath, whereby the entire bottle becomes slightly turbid. Initially, a small amount of white foam settles on the neck of the bottle, which is to settle prior to consumption. The bottle is now shaken lightly and briefly and is ready for use.

FIG. 1 electropharmacogram (1.0 mg/kg CBD), relative change pattern over time:

Time-dependent progression of the electric output (“spectral power”) (ordinate) in percent from the 45-minute long pre-drug base line (values) in four brain regions of freely moving rats in the presence of a vehicle (4 ml/kg). The frequency ranges are depicted as bars on the abscissa, specifically delta (1), theta (2), alpha1 (3), alpha2 (4), beta1a (5), beta1b (6) and gamma spectral power (7) from left to right, depending on the named brain region. The statistical significance is represented in stars compared to the control group (vehicle): *=p<0.05; **=p<0.01.

FIG. 2, electropharmacogram (5.0 mg/kg CBD), otherwise as in FIG. 1.

FIG. 3, electropharmacogram (15.0 mg/kg CBD), otherwise as in FIG. 1.

1.-11. (canceled)

12. A liquid formulation comprising cannabidiol (CBD) and water and at least one emulsifier for use in the application of a dosage of 1 mg/kg to 5 mg/kg (body weight) CBD in a human, wherein the cannabidiol is synthetic cannabidiol or purified cannabidiol from a cannabis extract.

13. The liquid formulation according to claim 12, comprising CBD in the range of 50 mg/L to 300 mg/L.

14. The liquid formulation according to claim 12, comprising CBD in the range of 50 mg/L to 100 mg/L.

15. The liquid formulation according to claim 12, wherein the emulsifier is a non-ionic, anionic, cationic or amphoteric emulsifier.

16. The liquid formulation according to claim 14, wherein the emulsifier is a non-ionic, anionic, cationic or amphoteric emulsifier.

17. The liquid formulation according to claim 12, wherein the emulsifier is selected from the group consisting of polysorbates, polyoxyethylene sorbitan fatty acid ester, rubbers, gum arabic, pectins, starches and modified starches, proteins and mixtures thereof.

18. The liquid formulation according to claim 16, wherein the emulsifier is selected from the group consisting of polysorbates, Tween 20, Tween 60, Tween 80, rubbers, gum arabic, pectins, starches, Purity Gum, caseinates from milk and mixtures thereof.

19. A beverage or base material comprising a liquid formulation comprising cannabidiol and water and at least one emulsifier, wherein the cannabidiol is synthetic cannabidiol or purified cannabidiol from a cannabis extract,

20. The beverage or base material according to claim 19, wherein the beverage or base material is selected from the group consisting of energy drink, mineral water, lemonade, tonic water, sports, mineral, fruit, fruit juice, milk, whey, soft drink, alcoholic beverage and as a drinking water preparation.

21. The beverage or base material according to claim 19, wherein the beverage or base material is selected from the group consisting of energy drink, mineral water, lemonade, tonic water, sports, mineral, fruit, fruit juice, milk, whey, soft drink, beer and as a drinking water preparation.

22. The beverage or base material according to claim 19, comprising 50 mg/L to 300 mg/L CBD.

23. The beverage or base material according to claim 22, comprising 50 mg/L to 100 mg/L CBD.

24. The beverage or base material according to claim 16, wherein the emulsifier is selected from the group consisting of polysorbates, polyoxyethylene sorbitan fatty acid ester, rubbers, gum arabic, pectins, starches and modified starches, proteins, caseinates, beeswax, lecithin, sterols and mixtures thereof.

25. The beverage or base material according to claim 16, wherein the emulsifier is selected from the group consisting of polysorbates, Tween 20, Tween 60, Tween 80, rubbers, gum arabic, pectins, starches and modified starches, proteins, caseinates, beeswax, lecithin, sterols and mixtures thereof.

26. A food or functional food comprising the beverage or base material according to claim 19.

27. A product comprising a liquid formulation comprising cannabidiol (CBD) and water and at least one emulsifier, comprising 50 mg/L to 300 mg/L CBD, for use for increased concentration, increased physical, athletic and cognitive performance and/or increased memory function, wherein the cannabidiol is synthetic cannabidiol or purified cannabidiol from a cannabis extract.

28. The product as claimed in claim 27, wherein the product is a pharmaceutical product or a dietary supplement comprising a liquid formulation comprising cannabidiol and water and at least one emulsifier, comprising 50 mg/L to 100 mg/L CBD, for use for increased concentration, increased physical, athletic and cognitive performance and/or increased memory function, wherein the cannabidiol is synthetic cannabidiol or purified cannabidiol from a cannabis extract.
